(19) United States
(12) Patent Application Publication
(10) Pub. No.: US 2014/0364687 A1
(43) Pub. Date: Dec. 11, 2014

(54) PENIS ENLARGER - BLANC SLIDER

(71) Applicant: CHARLES GERYESHELIAS BLANC, SR., Hialeah, FL (US)

(72) Inventor: CHARLES GERYESHELIAS BLANC, SR., Hialeah, FL (US)

(73) Assignee: Mr. Charles G. Blanc, SR., Hialeah, FL (US)

(21) Appl. No.: 14/328,739

(22) Filed: Jul. 11, 2014

(51) Int. Cl. A61F 5/41

(52) U.S. Cl. CPC A61F 5/41; A61F 2005/411
USPC 600/38

(57) ABSTRACT

At the present time did not find any patent list at Uspto but there is a model type that uses rods the old fashion way that requires an erection in order to have the penis stretched

### CROSS REFERENCE TO RELATED APPLICATIONS

This application is related to application Ser. No. 143187565

### FIELD OF THE INVENTION

This invention relates to an old penis extender were you require of the use of rods

### BACKGROUND OF THE INVENTION

The older invention called Penis extender that uses rods and must have a full erection in order to be able to stretch the penis certain people take medication drugs just to have an erection to be able to stretch the penis this present invention is a slider like penis enlarger you will find and see different views of the present invention were does not require any rods and don't even require a full erection in order to stretch the penis

### BRIEF SUMMARY OF THE INVENTION

In a first aspect this invention provides and offers a slider like with gaps were the pin like button locks in place making it easy and simple to use much saver than the old invention that uses rods and have to exchange to a different size all the time causing injury to ones self penis and having difficulty keeping an erection.

### OBJECT OF THE INVENTION

Is to provide comfort and safety of use

The same time gaining the size desired safely

not causing injury to one self by not having to change rods the old invention way

In addition to the old invention way this invention has two square tubes that slides in and out making it much simple and easy to use

Most importantly this invention avoids the need to have to take medication drugs in order to have an erection

### DETAILED DESCRIPTION OF THE INVENTION

This present invention is a penis stretcher that stretchs the muscle of the penis in order to enlarge the penis to a bigger size doing so using the two square tubes alike connected together with a penis stand that has a two short buttons that holds the belt straps and gives a option to use either the left button or right button and this invention is so easy and does not require an erection do to the fact as you can see in the drawings can be slided as close as you desire to the body causing no discomfort and as a slider sliding this invention in and out unlike the old invention that uses rods and require the use of rods for functioning without the rods the old invention is useless the present and new invention like discussed is a slider and easy to use

### BRIEF DESCRIPTION OF THE SEVERAL VIEWS OF THE DRAWING

FIG. 1 01 is a hollow gap in the belt strap to snap in one of the two short buttons on the penis stand 02 is the edge of the belt strap that is needed to put in from behind on the penis stand in order for the belt strap not to slip from the penis stand

FIG. 2 03 is the belt that you place through the penis stand in order to stap and tighten the penis head to prepare the penis for stretching

FIG. 3 03 is the same penis belt showing in a different view explaining certain parts of the present invention in order to easy to understand the invention when looking at the drawings

FIG. 4 03 is same as 03 except in a different view

FIG. 5 03 is also the same except in a different view

FIG. 6 04 is the metal piece spring like that is connected to 05 U is a bendable metal that allows the button to be pushed in and releases back do to the U is also spring like 06 is a straight metal piece that is connected to the pin like button 07 that with comfort and ease 07 can be pushed and the present invention can be slided in and out

FIG. 7 07 Is the pin like button as a whole piece

FIG. 8 07 Is the pin like button in a left view look

FIG. 9 Is the plastic cap that go inside 13 to cover the mechanism

FIG. 10 Is the same cap in a left view

FIG. 11 its the same cap in a bottom view

FIG. 12 its the same cap in a right view

FIG. 13 Is a larger cap that goes on the outside 18 sliding tubes

FIG. 14 Is the same large cap in a left view

FIG. 15 Is the same large cap in a bottom view

FIG. 16 is the present invention a solid large ring 11 to place around the penis and 10 is the hollow space were you place the unit followed by 13 a two hollow tube were the 16 penis stand slides in and out followed by 07 the push button spring like that allows you to lock the tubes in any of the gaps followed by a . 08 the small caps

FIG. 17 similar to FIG. 16 except in a right view

FIG. 18 similar to FIG. 16 in a top view

FIG. 19 similar to FIG. 16 in a left view

FIG. 20 Is 16 penis stand were you place the penis 14 the short buttons that the belt hooks on in order not to slip with 15 a hollow space were the 02 the back of the belt hold on so the belt does not slip 17 is the gaps were 07 the pring like push button slides through to lock the unit 18 is the two tubes with gaps that slides into 13 the main unit other two hollow tubes followed by 09 the large caps that protects the end of the unit

FIG. 21 Is the same as FIG. 20 except in a right view

FIG. 22 Is the same as FIG. 20 except in a top view

FIG. 23 Is the same as FIG. 20 except in a left view

FIG. 24 is the present invention a solid large ring 11 to place around the penis and 10 is the hollow space were you place the unit followed by 13 a two hollow tube were the 16 penis stand slides in and out followed by 07 the push button spring like that allows you to lock the tubes in any of the gaps followed by a small cap Followed by 16 penis stand were you place the penis 14 the short buttons that the belt hooks on in order not to slip with 15 a hollow space were the 02 the back of the belt hold on so the belt does not slip 17 is the gaps were 07 the pring like push button slides through to lock the unit 18 is the two tubes with gaps that slides into 13 the main unit other two hollow tubes followed by 09 the large caps that protects the end of the unit

FIG. 25 is the same as FIG. 24 except in a right view

FIG. 26 is the same as FIG. 24 except in a top view

FIG. 27 in this view the invention discusses the function of the mechanism of the spring like push button within the tubes in the unit as you see in the view of FIG. 27 04 the end of the spring push buttons with 05 the U shape spring and how it is bend being pushed in to slide the tubes in and out 06 is a solid piece that connects the spring 04 and 05 into 07 the spring push button now in relates to the spring push button you will notice that the tubes are pushed far out at a different gap do to that the spring push button is pushed in

FIG. 28 07 the spring like push button is still pushed in so you can see the tubes are pushed in instead of out and the penis stand is closer to the body in order to stretch the penis

FIG. 29 07 the spring like push button is locked as you see 04 05 06 are pulled back do to releasing the button at one of the gap spots

FIG. 30 Showing the 03 belt for the unit and its contents as of 01 one of holes in the belt that holds to the short buttons so 03 the belt don't slip showing 02 the end of the belt that has an edge that allows the belt to stay holding to the penis stand from slipping followed by 15 the empty cut space for the belt insert through Followed by 07 the spring push button as seen in this view the button is pushed in

FIG. 31 in this view the new invention you can see an easy illustration how 03 the belt is being placed and tighten before the penis head 20 simple showing the complete bell and its functions how is 01 one of the holes of the belt is in 14 one of the short buttons on the right strapped the penis head also shows how 19 the testicules are free and not being touched by the unit and 20 the penis straight and placed on the stand and 07 the spring like push button is pushed in and the tubes are movable in and out

FIG. 32 In this view you will see the same as FIG. 31 except the belt is being strapped on the left side of the stand showing that 14 the two short buttons gives an option tighten the belt either left or right side and if you noticed 07 the spring like push button is still pushed in and the tubes are movable in and out

FIG. 33 the same as FIG. 32 is the same 03 the belt is strapped on left side of the stand showing that 14 the two short buttons gives an option to tighten the belt either left or right side and if you noticed 07 the spring like push button is released in this view and the unit is locked in one of the gaps

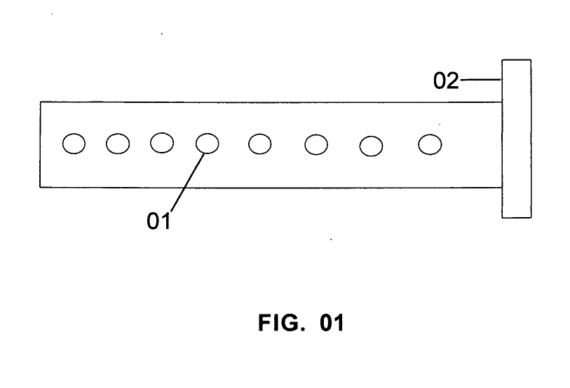

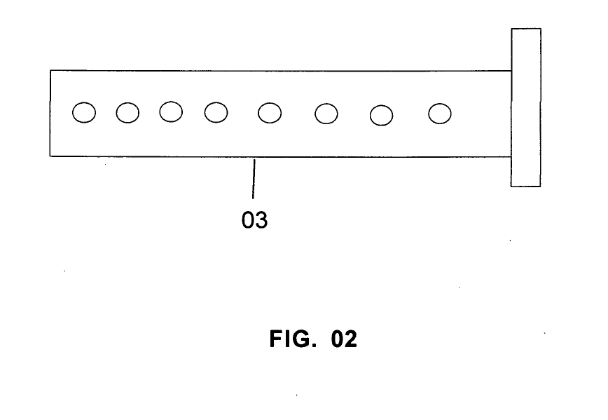

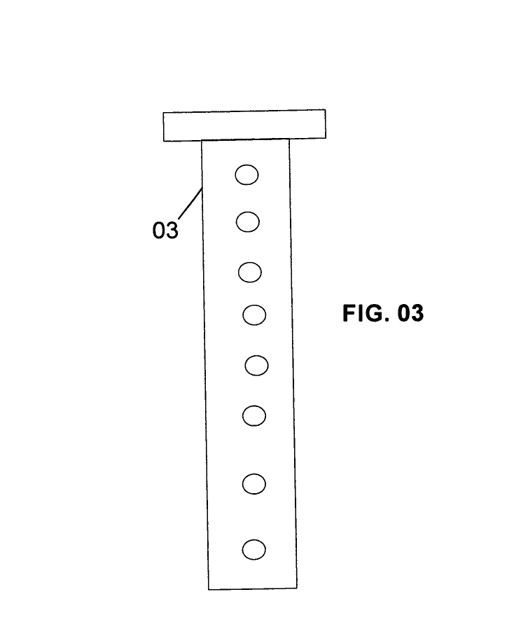

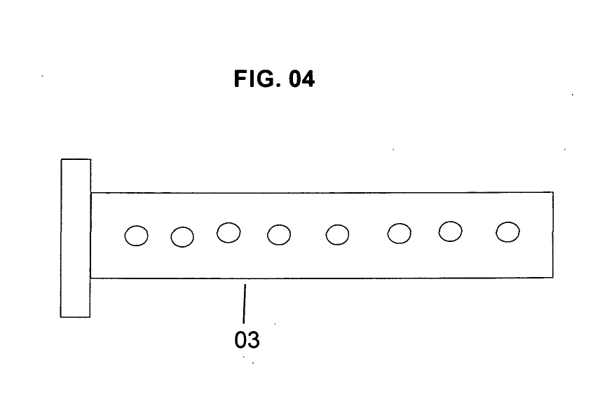

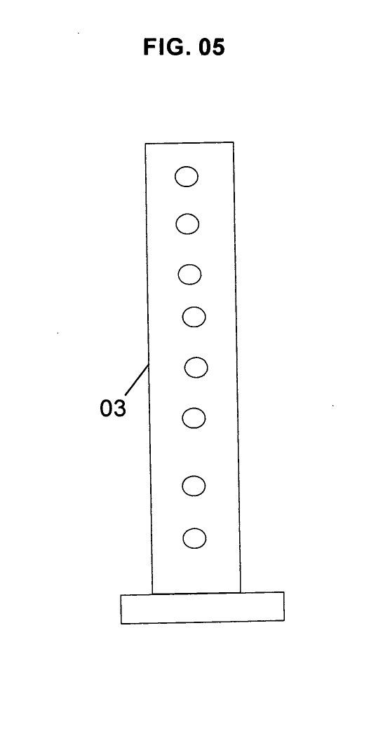

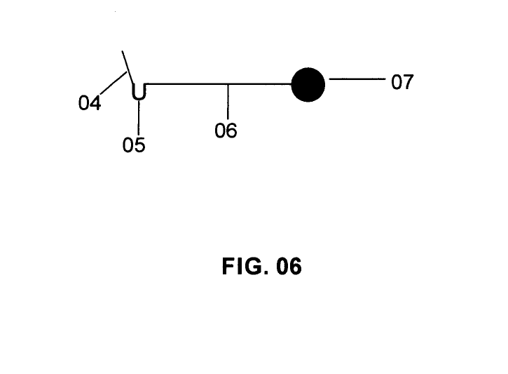

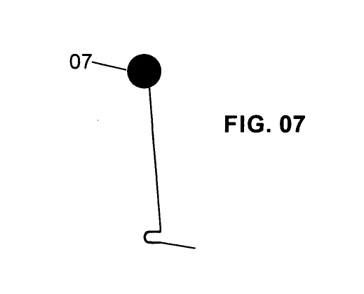

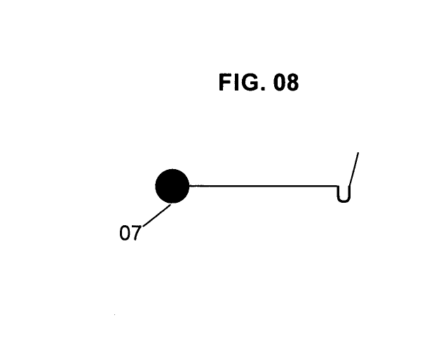

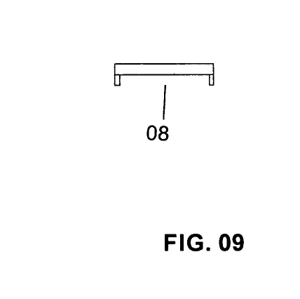

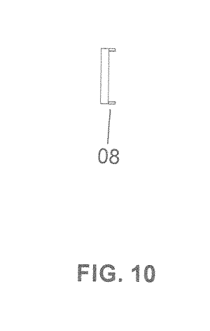

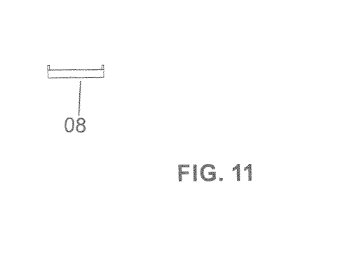

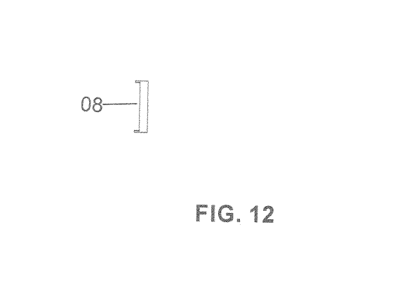

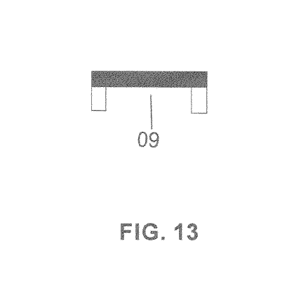

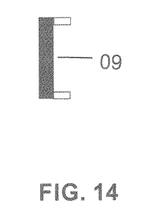

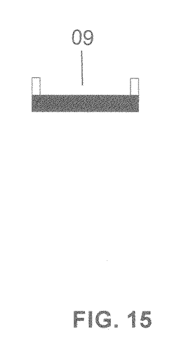

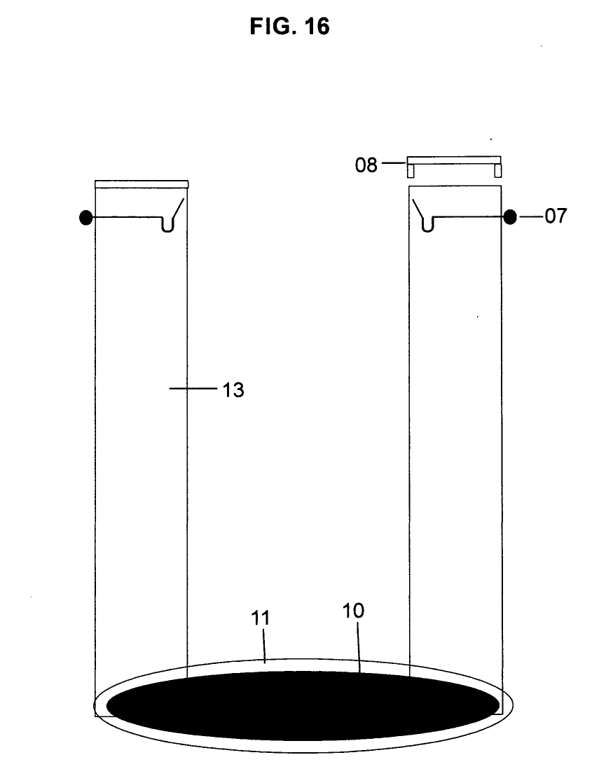

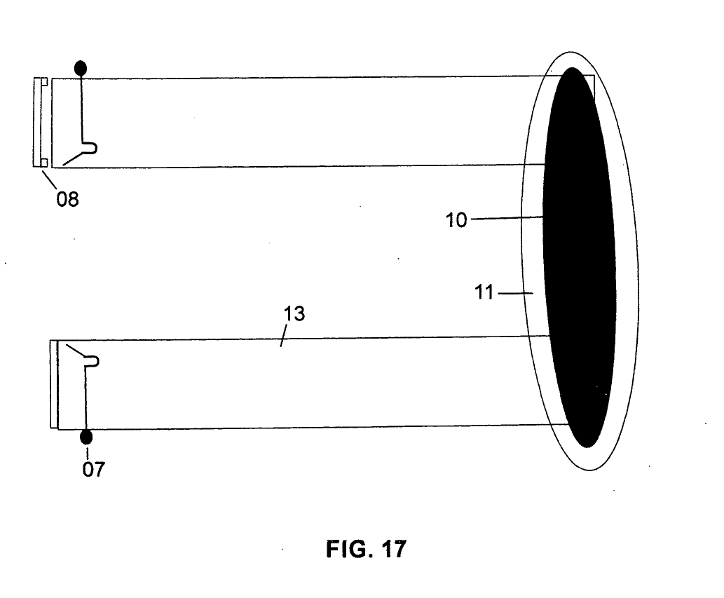

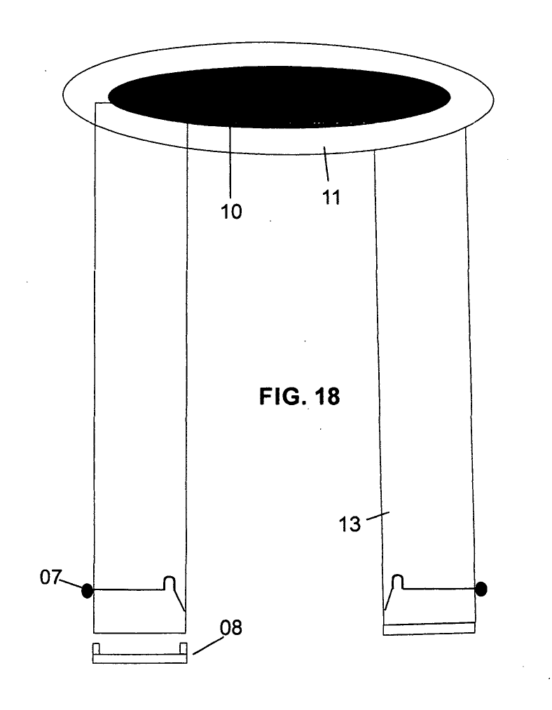

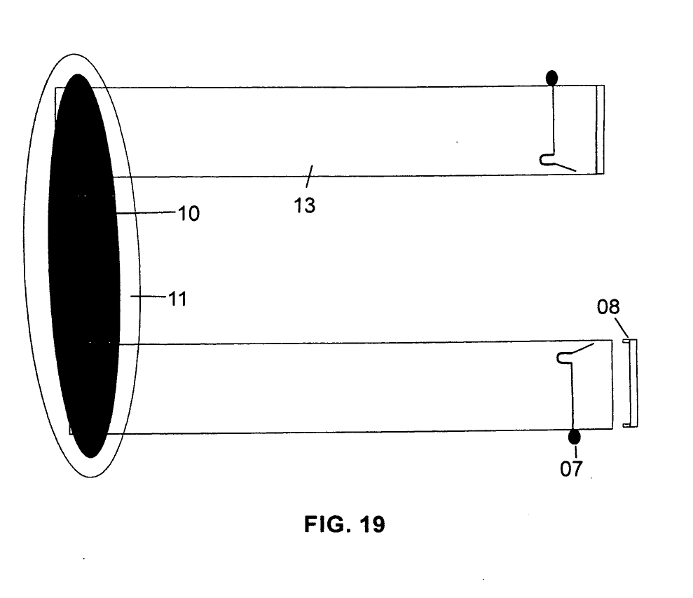

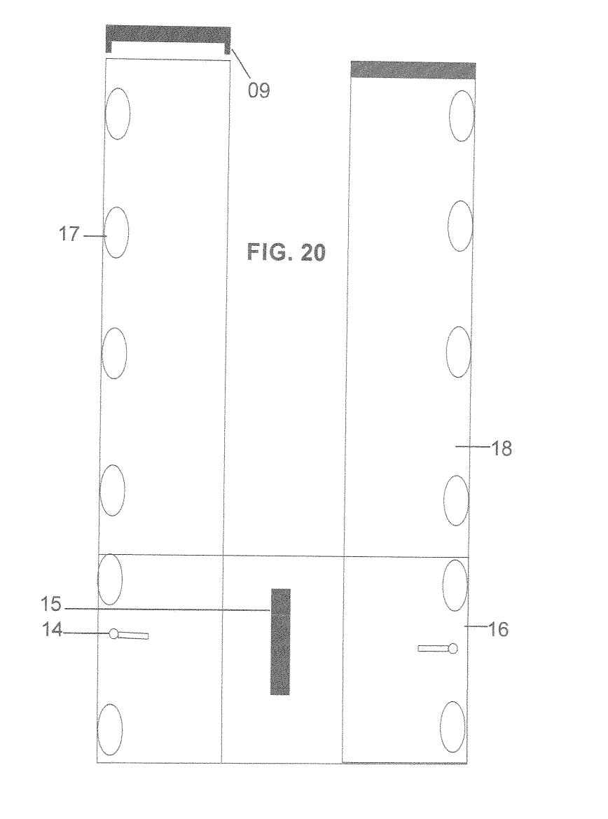

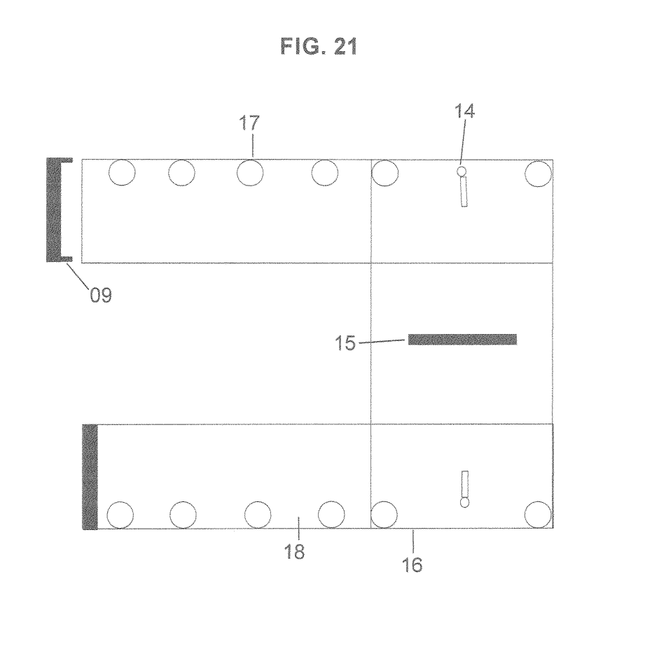

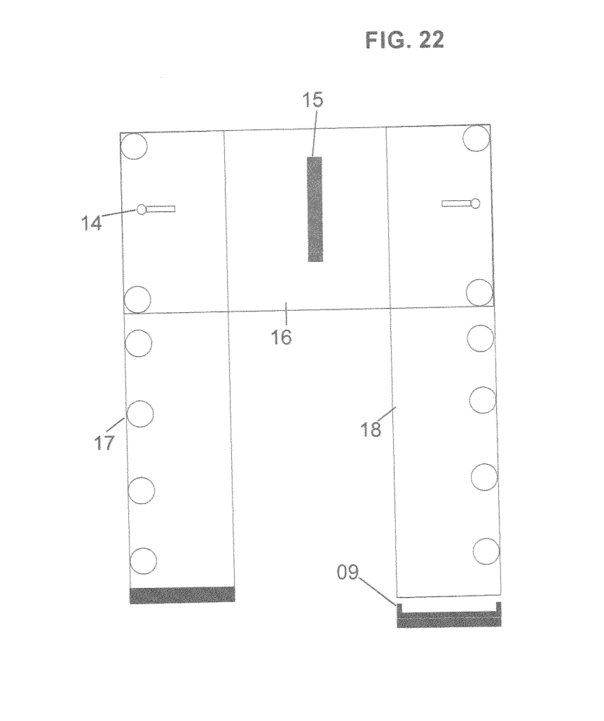

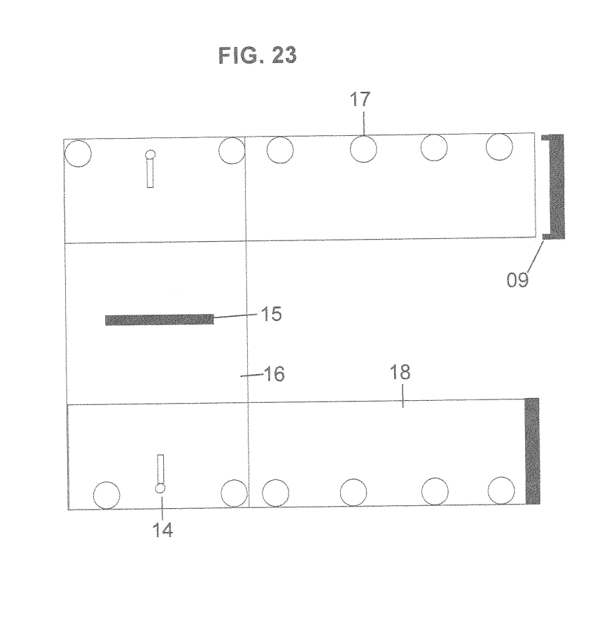

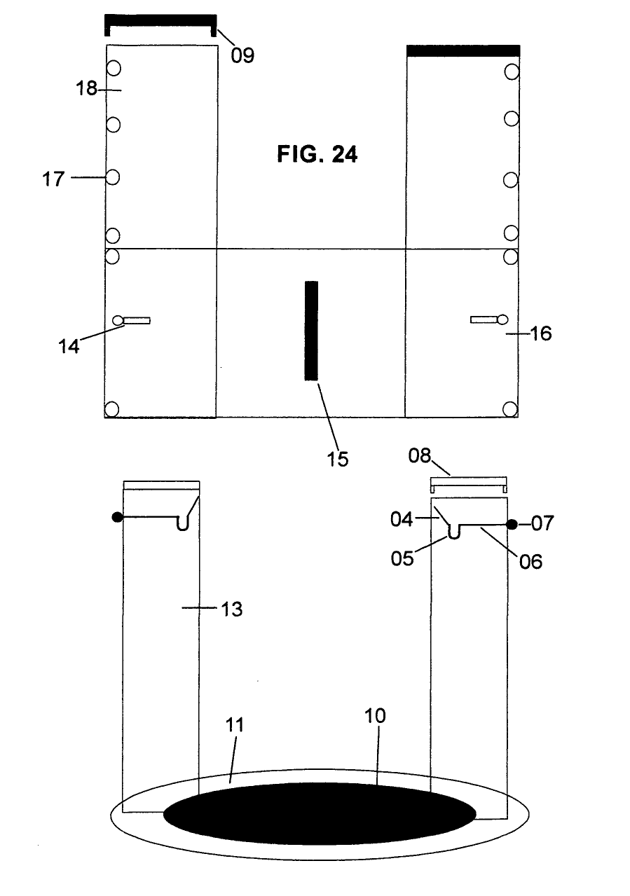

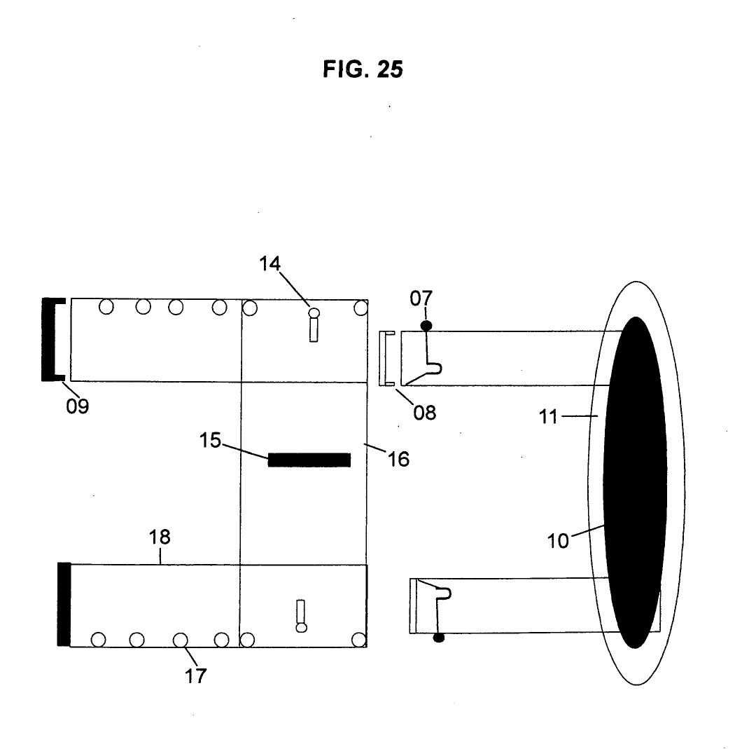

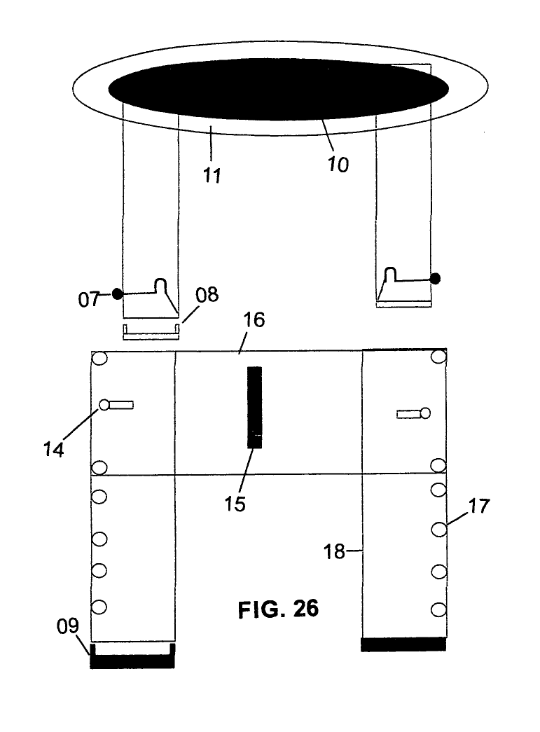

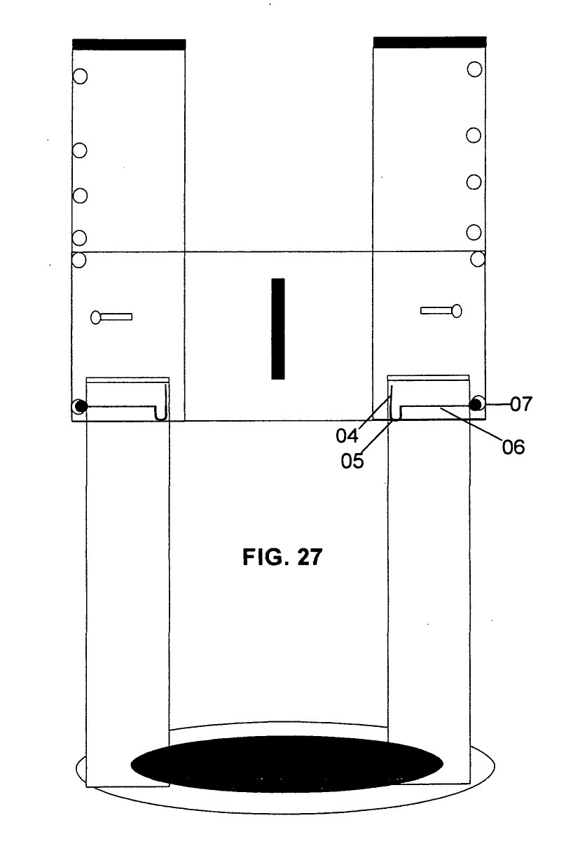

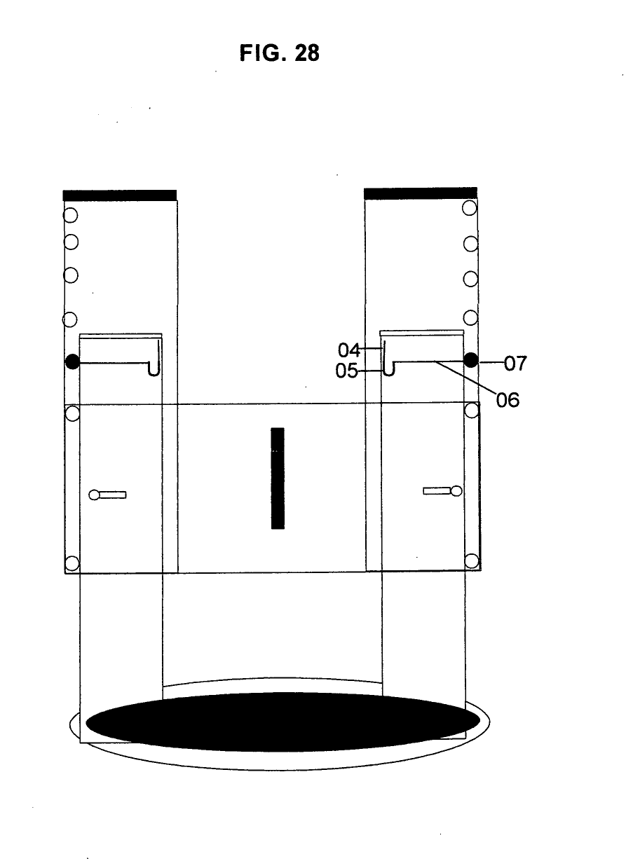

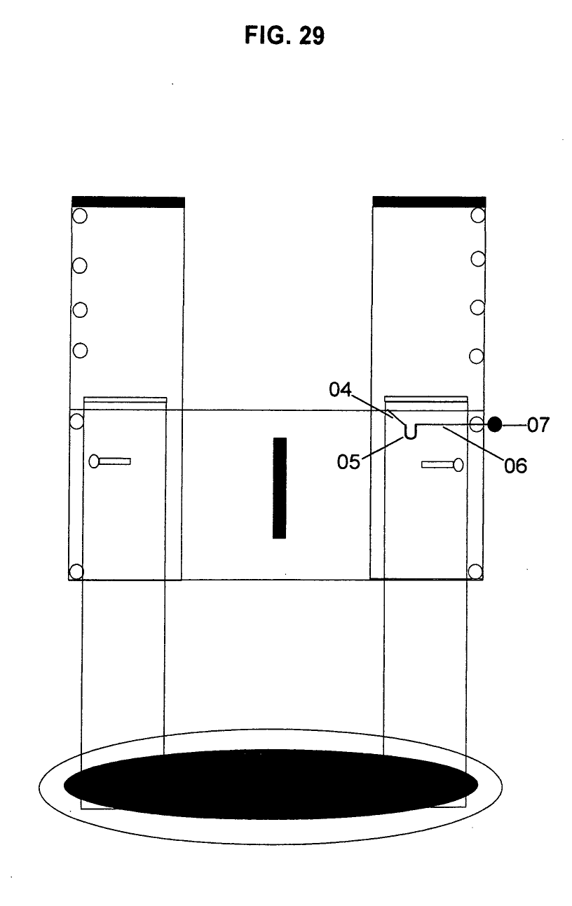

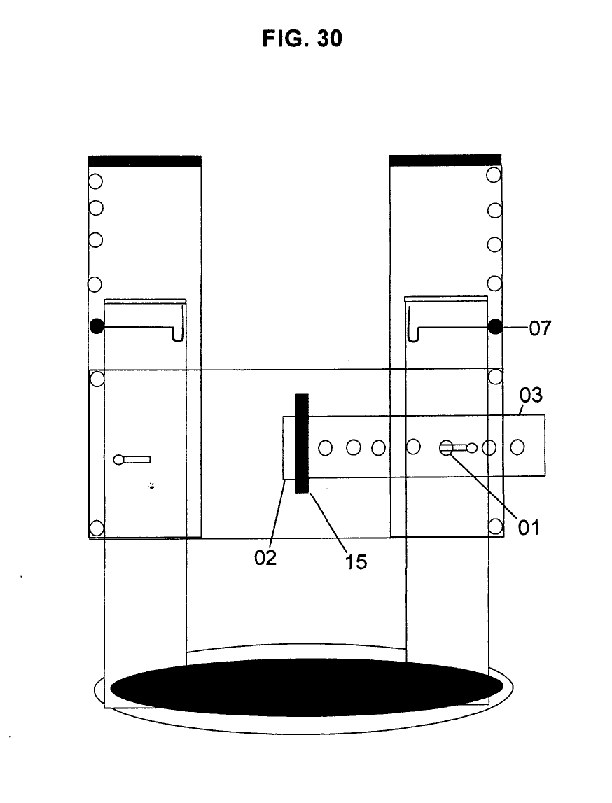

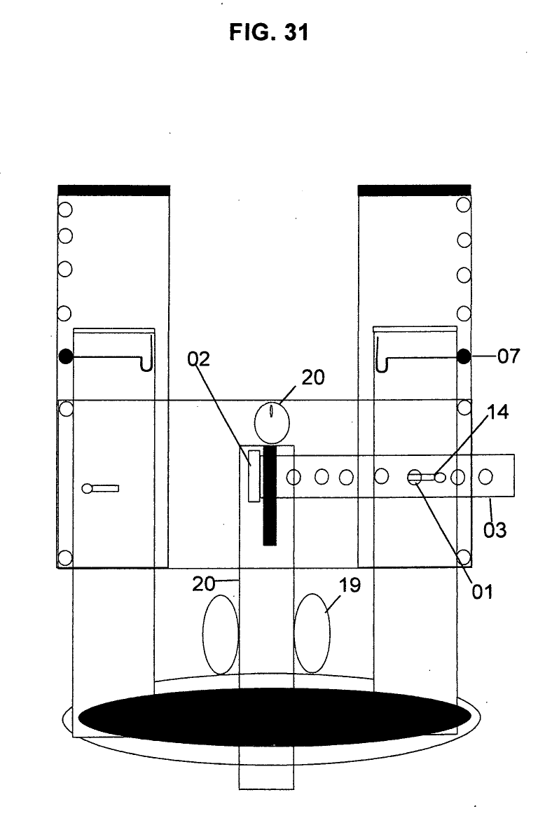

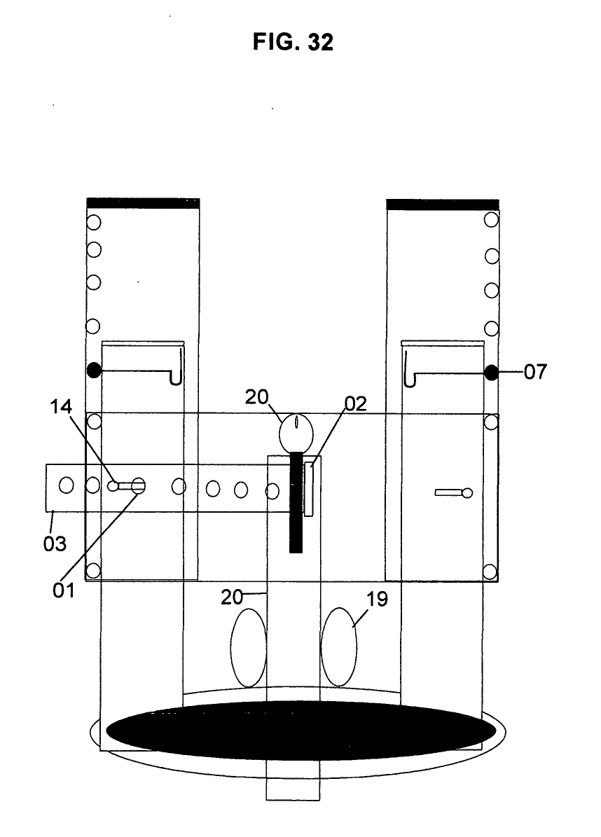

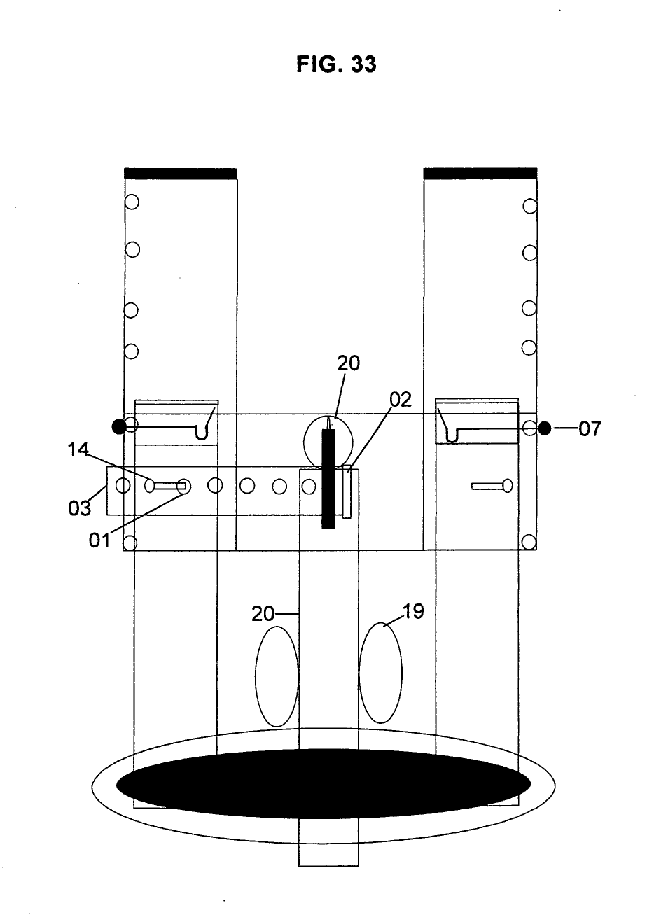

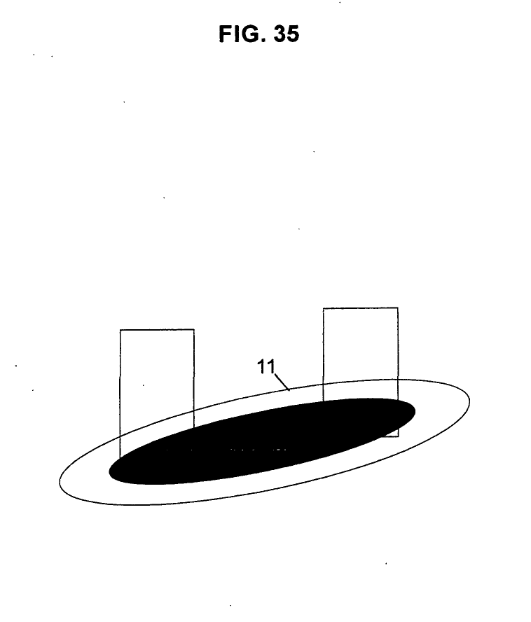

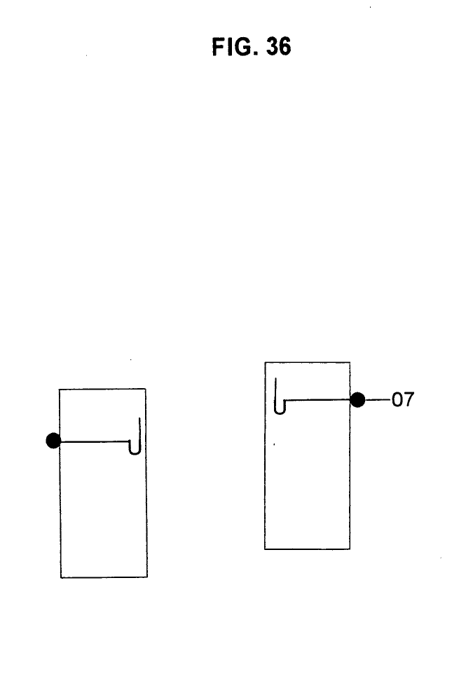

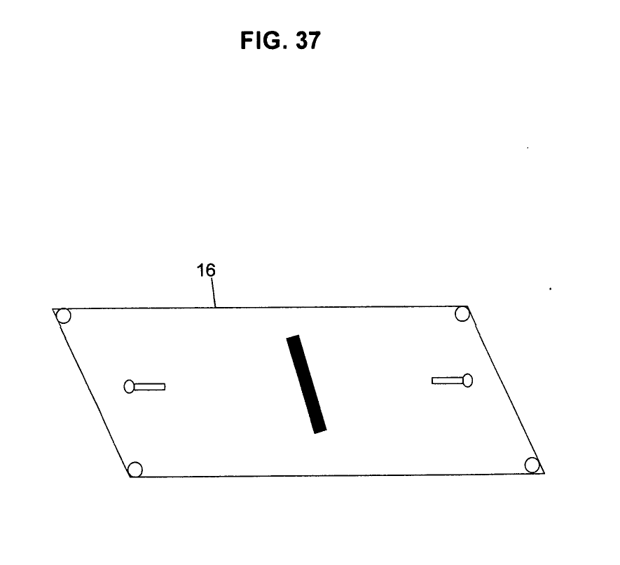

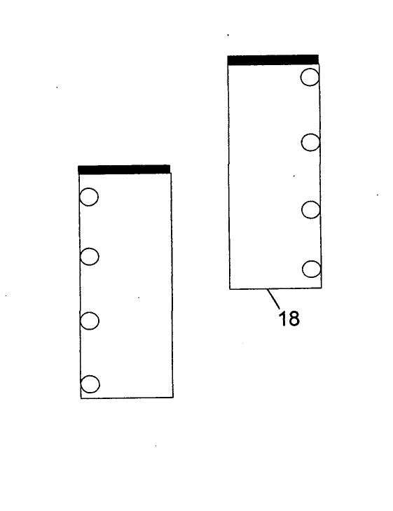

1. This present invention is a slider using square tubes alike on both ends and a penis stand that connected in the middle and have two short buttons that lets you use the belt on either button left or right the penis stand together they offer a sliding mechanism that can be slided in and out and most importantly the push button that can be pushed in that locks the tubes in place avoiding the use of rods and safer
